(19) United States
(12) Patent Application Publication
(10) Pub. No.: US 2013/0288932 A1
(43) Pub. Date: Oct. 31, 2013

(54) METHODS OF CRYODESICCATING A BROTH COMPRISING A BIOPOLYMER OF AN EXOPOLYSACCHARIDE

(71) Applicants: Andrew A.F. Mackenzie, Dyce (GB); Eric Davidson, Dyce (GB)

(72) Inventors: Andrew A.F. Mackenzie, Dyce (GB); Eric Davidson, Dyce (GB)

(73) Assignee: HALLIBURTON ENERGY SERVICES, INC., Houston, TX (US)

(21) Appl. No.: 13/458,235

(22) Filed: Apr. 27, 2012

(51) Int. Cl. C09K 8/035; C12P 19/04; C12P 19/06; C12P 19/10; C12P 19/08; C04B 16/02; C08B 15/00; C08B 37/08; C08B 37/04; C09K 8/62; C04B 16/00; C08B 37/00; C08B 37/02

(52) U.S. Cl. 507/112; 536/123.1; 435/101; 435/104; 435/102; 435/103; 536/123.12; 536/55.2; 536/56; 536/55.1; 536/20; 536/123; 435/84; 507/110; 435/85; 507/211; 507/213; 507/214; 106/804

(57) ABSTRACT

According to an embodiment, a method of providing a biopolymer comprises: cryodesiccating a broth, wherein the broth comprises: (A) a biopolymer, wherein the biopolymer is an exopolysaccharide; and (B) a liquid growth medium. According to certain embodiments, the biopolymer is produced by the fermentation of at least one carbohydrate via a microbe. According to an embodiment, the microbe is a bacterium. According to another embodiment, the microbe is a fungus.

### TECHNICAL FIELD

Methods of providing a biopolymer are provided. The biopolymer can be an exopolysaccharide. According to certain embodiments, the biopolymer is included in a broth. The broth is cryodesiccated to preserve the original configuration of the biopolymer, resulting in improved properties of the biopolymer.

### SUMMARY

According to an embodiment, a method of providing a biopolymer comprises: cryodesiccating a broth, wherein the broth comprises: (A) a biopolymer, wherein the biopolymer is an exopolysaccharide; and (B) a liquid growth medium.

### DETAILED DESCRIPTION OF THE INVENTION

As used herein, the words “comprise,” “have,” “include,” and all grammatical variations thereof are each intended to have an open, non-limiting meaning that does not exclude additional elements or steps.

Polymers are used in a variety of industries. For example, polymers are commonly used in pharmaceutical formulations, cosmetics, agricultural products, textile printing, pastes, ceramic glazes, slurry explosive formulations, and rust removers. Moreover, polymers are commonly used in the oil and gas industry.

A polymer is a large molecule composed of repeating units, typically connected by covalent chemical bonds. A polymer is formed from monomers. During the formation of the polymer, some chemical groups can be lost from each monomer. The piece of the monomer that is incorporated into the polymer is known as the repeating unit or monomer residue. The backbone of the polymer is the continuous link between the monomer residues. The polymer can also contain functional groups connected to the backbone at various locations along the backbone. Polymer nomenclature is generally based upon the type of monomer residues comprising the polymer. A polymer formed from one type of monomer residue is called a homopolymer. A copolymer is formed from two or more different types of monomer residues. The number of repeating units of a polymer is referred to as the chain length of the polymer. The number of repeating units of a polymer can range from approximately 11 to greater than 10,000. In a copolymer, the repeating units from each of the monomer residues can be arranged in various ways along the polymer chain. For example, the repeating units can be random, alternating, periodic, or block. The conditions of the polymerization reaction can be adjusted to help control the average number of repeating units (the average chain length) of the polymer.

A polymer has an average molecular weight, which is directly related to the average chain length of the polymer. The average molecular weight of a polymer has an impact on some of the physical characteristics of a polymer, for example, its solubility in water, its viscosity, and its biodegradability. For a copolymer, each of the monomers will be repeated a certain number of times (number of repeating units). The average molecular weight for a copolymer can be expressed as follows:

Avg. molecular weight=(M.W.m₁*RU m₁)+(M.W.m₂*RU m₂)

where M.W.m₁ is the molecular weight of the first monomer; RU m₁ is the number of repeating units of the first monomer; M.W.m₂ is the molecular weight of the second monomer; and RU m₂ is the number of repeating units of the second monomer. Of course, a terpolymer would include three monomers, a tetra polymer would include four monomers, and so on.

Biopolymers are polymers made by living organisms. Biopolymers generally include polynucleotides, polypeptides, and polysaccharides. Xanthan is an example of a polysaccharide biopolymer. A polysaccharide biopolymer can be made by the fermentation of a sugar (i.e., a carbohydrate, for example a monosaccharide, disaccharide or polysaccharide) via one or more microorganisms, also known as a microbe. Xanthan, for example, can be formed by the fermentation of glucose, sucrose, or lactose by the Xanthomonas campestris bacterium. Extracellular polysachharide substances (EPS) or exopolysaccharides are high molecular weight polymers that are composed of sugar residues and are extracellular polymers secreted outside the cell walls of a microorganism. As used herein, a “high molecular weight polymer” is a polymer having a molecular weight of greater than 50,000. Exopolysaccharides and their corresponding microorganism include, but are not limited to, acetan (Acetobacter xylinum), alginate (Azotobacter vinelandii), cellulose (Acetobacter xylinum), chitosan (Mucorales spp.), curdlan (Alcaligenes faecalis var. myxogenes), cyclosophorans (Agrobacterium spp., Rhizobium spp. and Xanthomonas spp.), dextran (Leuconostoc mesenteroides, Leuconostoc dextranicum and Lactobacillus hilgardii), emulsan (Acinetobacter calcoaceticus), galactoglucopolysaccharides (Achromobacter spp., Agrobacterium radiobacter, Pseudomonas marginalis, Rhizobium spp. and Zooglea' spp.), gellan (Aureomonas elodea and Sphingomonas paucimobilis), glucuronan (Sinorhizobium meliloti), N-acetyl-glucosamine (Staphylococcus Epidermidis), N-acetyl-heparosan (Escherichia coli), hyaluronic acid (Streptococcus equi), indican (Beijerinckia indica), kefiran (Lactobacillus hilgardii), lentinan (Lentinus edodes), levan (Alcaligenes viscosus, Zymomonas mobilis, Bacillus subtilis), pullulan (Aureobasidium pullulans), scleroglucan (Sclerotium rolfsii, Sclerotium delfinii and Sclerotium Glucanicum), schizophyllan (Schizophylum commune), stewartan (Pantoea stewartii subsp. stewartii), succinoglycan (Alcaligenes faecalis var myxogenes, Sinorhizobium meliloti), xanthan (Xanthomonas campestris), and welan (Alcaligenes spp.).

Biopolymer formation generally involves several steps. A small amount of a preserved microbial culture is expanded by growth on solid surfaces or in liquid medium to obtain the inoculum for bioreactors. The microbe, a carbohydrate, and a liquid growth medium are added to the bioreactor to allow the carbohydrate to ferment. Generally, trace elements, vitamins, and a defined carbon source and nitrogen source are required by the microbe. Glucose or glycerol are often used as carbon sources, and ammonium salts or nitrates as inorganic nitrogen sources. At the end of the fermentation process, the fermented broth contains the polymer, microbial cells, and many other chemicals. The main steps of a traditional recovery process of the polymer include deactivation and removal (or lysis) of the microbial cells, precipitation of the polymer, dewatering, drying, and milling.

Insoluble products are generally removed from the fermentation broth via one or more of the following: filtration, centrifugation, sedimentation, precipitation, flocculation, electro-precipitation, and gravity settling. For example, precipitation of a polymer is achieved by decreasing the solubility of the dissolved colloid using methods such as addition of salts, addition of water-miscible non-solvents, and concentration by evaporation. The most common technique used for the primary isolation and purification of polysaccharides is precipitation using water-miscible non-solvents such as alcohols. The lower alcohols (methanol, ethanol, and isopropanol) and acetone, which are non-solvents for the polysaccharide, can be added to the fermentation broth not only to decrease the solubility until phase separation occurs, but can also wash out impurities such as colored components, salts, and microbial cells. After removal of the insoluble products, the polymer is generally de-watered and dried. The dried polymer can be milled and placed into a container.

Polymers are commonly used in the oil and gas industry. Polymers can be added to a variety of wellbore treatment fluids such as drilling fluids and completion fluids. Commonly used biopolymers include xanthan, diutan, scleroglucan, succinoglycan, cellulose, chitosan, dextran, acetan, alginate, curdlan, cyclosophorans, emulsan, galactoglucopolysaccharides, gellan, glucuronan, N-acetyl-glucosamine, N-acetyl-heparosan, hyaluronic acid, indican, kefiran, lentinan, levan, pullulan, schizophyllan, stewartan and welan. The addition of a polymer can increase the viscosity of the fluid and/or increase the suspending properties of the fluid. Viscosity is a measure of the resistance of a fluid to flow, defined as the ratio of shear stress to shear rate. Viscosity can be expressed in units of (force*time)/area. For example, viscosity can be expressed in units of dyne*s/cm² (commonly referred to as Poise (P)), or expressed in units of Pascals/second (Pa/s). However, because a material that has a viscosity of 1 P is a relatively viscous material, viscosity is more commonly expressed in units of centipoise (cP), which is 1/100 P. The suspending property of a fluid is related to the ability of the fluid to suspend insoluble particulates within the fluid and prevent the particulates from settling out of the fluid.

Some polymers can swell in the presence of a water- or oil-based liquid. As used herein, the term “swellable” means a polymer that can absorb a liquid or incorporate a liquid into its microstructure and swell to a size that is bigger than the size of the pre-swelled polymer. As used herein, the term “water based” means a fluid that contains an aqueous liquid. The aqueous liquid can be freshwater, saltwater, or a brine. As used herein, the term “oil based” means a fluid that contains a hydrocarbon liquid.

However, it can be difficult to add a polymer to a liquid because the polymer can form fish eyes in the liquid. A fish eye generally occurs during the process of blending the swellable polymer with the liquid. Fish eyes are balls of unhydrated polymer surrounded by a gelatinous covering of hydrated polymer. Fish eyes prevent the liquid from contacting the interior of the fish eye and the unhydrated polymer contained therein. Fish eyes can be difficult to break apart once formed. Therefore, a polymer is generally added to the liquid at a relatively slow rate in order to help minimize fish eye formation. However, even with slow addition of the polymer, fish eye formation can still be problematic. Moreover, having to add the polymer to the liquid at a slow rate means that valuable time is spent forming the fluid.

The formation of fish eyes can occur due to the configuration of the polymer itself, among other things. During production of the biopolymer via the microbe, the original configuration of the polymer is generally long chains. However, during the recovery of a biopolymer from the fermentation broth, the polymer chains generally become tangled. During recovery of xanthan for example, the xanthan molecules generally have two conformations, helix and random coil. The tangled configuration of the polymer molecules means that less surface area is available to contact the liquid and thus, causes non-uniform swelling of the polymer.

It has been discovered that a cryodesiccated, biopolymer broth can be used in a variety of applications. The freeze-dried biopolymer can exhibit improved characteristics, such as a better configuration compared to a biopolymer that is recovered via traditional methods of separation, de-watering, and drying. The freeze-dried polymer can: swell faster; be added at a higher rate; and hydrate more fully compared to biopolymers recovered via the traditional methods.

According to an embodiment, a method of providing a biopolymer comprises: cryodesiccating a broth, wherein the broth comprises: (A) a biopolymer, wherein the biopolymer is an exopolysaccharide; and (B) a liquid growth medium.

The broth comprises the biopolymer. As used herein, the term “biopolymer” means a polymer formed by the fermentation of at least one carbohydrate via a microorganism. The microorganism, commonly called a microbe, can be a bacterium or a fungus (e.g., a yeast or a mold). A carbohydrate is an organic compound with the empirical formula Cₘ(H₂O)ₙ. The carbohydrate can be a monosaccharide, disaccharide, oligosaccharide or polysaccharide. According to an embodiment, the carbohydrate is a monosaccharide or a disaccharide. The monosaccharide can contain at least 3 carbon atoms. The monosaccharide can be, without limitation, glucose, fructose, galactose, mannose or xylose. A disaccharide is a carbohydrate formed via a condensation reaction wherein two monosaccharides are joined together with the loss of a water (H₂O) molecule. The disaccharide can be, without limitation, sucrose, lactulose, lactose, maltose, trehalose or cellobiose. The biopolymer can also be formed by the fermentation of two or more carbohydrates via the microbe. The two or more carbohydrates can be the same type or different types. For example, both carbohydrates can be a monosaccharide. According to this example, the first carbohydrate can be glucose and the second carbohydrate can be fructose. Of course different combinations of monosaccharides can be created. By way of another example, the first carbohydrate can be a monosaccharide and the second carbohydrate can be a disaccharide.

The biopolymer is an exopolysaccharide. The exopolysaccharide can comprise a monosaccharide. The exopolysaccharide can also comprise one or more non-carbohydrate substituents. The non-carbohydrate substituents can include, without limitation, acetate, pyruvate, succinate, and phosphate. The biopolymer can be a homopolymer. An example of a homo-biopolymer is scleroglucan. The biopolymer can also be a copolymer. Xanthan is an example of a copolymer and includes a main chain composed of glucose units with a trisaccharide side chain made up of α-D-mannose with an acetyl group, β-D-glucuronic acid and a terminal β-D-mannose unit linked to a pyruvate group.

The biopolymer can be selected from the group consisting of, acetan, alginate, cellulose, chitosan, curdlan, cyclosophorans, dextran, emulsan, galactoglucopolysaccharides, gellan, glucuronan, N-acetyl-glucosamine, N-acetyl-heparosan, hyaluronic acid, indican, kefiran, lentinan, levan, pullulan, scleroglucan, schizophyllan, stewartan, succinoglycan, xanthan, and welan.

The biopolymer is formed by the fermentation of the at least one carbohydrate via a microbe. Fermentation is the process whereby oxidation of the carbohydrate via the microbe occurs. The microbe can be selected based on the type of desired biopolymer. For example, if xanthan is the desired biopolymer, then the microbe selected would be the bacterium Xanthomonas campestris. The microbe can be a bacterium. The bacterium can be, without limitation, Acetobacter xylinum, Azotobacter vinelandii, Alcaligenes faecalis var. myxogenes, Agrobacterium spp., Rhizobium spp., Xanthomonas spp., Leuconostoc mesenteroides, Leuconostoc dextranicum, Lactobacillus hilgardii, Acinetobacter calcoaceticus, Achromobacter spp., Agrobacterium radiobacter, Pseudomonas marginalis, Rhizobium spp., Zooglea' spp., Aureomonas elodea, Sphingomonas paucimobilis, Sinorhizobium meliloti, Staphylococcus epidermidis, Escherichia coli, Streptococcus equi, Beijerinckia indica, Alcaligenes viscosus, Zymomonas mobilis, Bacillus subtilis, Pantoea stewartii subsp. stewartii, Xanthomonas campestris, or Alcaligenes spp. The microbe can be a fungus. The fungus can be, without limitation, Mucorales spp., Lentinus edodes, Aureobasidium pullulans, Sclerotium rolfsii, Sclerotium delfinii, Sclerotium glucanicum, or Schizophylum commune.

The methods can further include the following steps, wherein the following steps are performed prior to the step of cryodesiccating, unless otherwise stated. It is to be understood that not all of the following steps need to be performed. Some of the steps can be performed while other steps are not performed. Whether a particular step is performed can be determined based on the availability of the material discussed in the step. By way of example, if a step is allowing the fermentation of the carbohydrate, then this step may not be performed if a source of fermented broth is available.

The methods can include preserving the microbe, obtaining the microbe, and/or growing the microbe. One of ordinary skill in the art will be able to determine the best method for preserving the microbe and growing the microbe. Some of the factors for determining the best growth of the microbe can include the type of bioreactor used, the mode of operation (e.g., batch or continuous), the growth medium composition, and the culture conditions (e.g., temperature, pH, and dissolved oxygen concentration).

The methods can further include the step of producing the biopolymer. The step of producing can include forming the broth. The step of producing can include placing at least the microbe, the carbohydrate, a nitrogen source, and the liquid medium in a bioreactor. The nitrogen source can be, without limitation, ammonium salts, amino acids, or very occasionally, a nitrate. The liquid medium can include water. The liquid medium can be a nutrient broth. The liquid medium can be a defined medium. A “defined medium” is a medium with known quantities of ingredients. Micronutrients, for example potassium, magnesium, sulphur, molybdenum, or the salts of potassium, iron, and calcium, can be present in the liquid medium. The micronutrients can vary and can be selected based on the microbe selected. Trace elements can also be included in the broth. Moreover, other ingredients can be added to the broth in order for the desired biopolymer to be formed. For example, an ingredient can be added in order to provide a monomer residue or functional group making up the biopolymer. The concentration of micronutrients can have an effect on the composition of the biopolymer. For example, in the case of xanthan, the micronutrient concentration can have an effect on the acetate, uronic acid, and pyruvate content of the molecules.

Production of the biopolymer can be accomplished via fermentation of the carbohydrate via the microbe. The biopolymer can be produced extracellularly by the microbe into the liquid medium. The fermentation can be accomplished in a batch-wise, step-wise, or continuous fashion. Various parameters can be controlled during the fermentation process. The various parameters controlled can include pH, aeration of the broth, temperature, agitation of the broth, and dissolved oxygen concentration. The optimal value of each parameter can be selected based on the type of microbe selected. By way of example, for xanthan production, the optimal fermentation temperature is between 75 and 80° F. (24 and 27° C.) and the optimal pH is between 7 and 8. The methods can also include the step of allowing the fermentation process to continue for a desired amount of time. The desired amount of time can be the time necessary to produce a desired quantity of the biopolymer. The methods can also include the step of stopping the fermentation process. The fermentation can be stopped in a variety of ways and can commonly be performed via pasteurization of the fermentation broth.

According to an embodiment, the broth is a fermentation broth. The broth includes the biopolymer and the liquid medium. The broth can also include microbial cells and nutrients, such as the nitrogen source, micronutrients, and trace elements. The methods can further include the step of deactivation and/or removal of any microbial cells that may be remaining in the broth after the formation of the biopolymer. Deactivation can be performed, for example, by causing lysis of the cells. Lysis of the cells can be accomplished by a variety of mechanisms, for example via the addition of an enzyme to the broth. Removal of the microbial cells can be accomplished by a variety of mechanisms, for example via centrifugation or filtration. According to an embodiment, the microbial cells are deactivated and/or removed from the broth in a manner that does not degrade the biopolymer. The methods can further include the step of adding a biocide to the broth in order to avoid microbial degradation. According to an embodiment, the broth contains the biopolymer in its original configuration. Preferably, the original configuration is a long chain (i.e., the configuration of the biopolymer is not tangled or randomly coiled). If the methods include the step of removing the microbial cells from the broth, then preferably the step of removing preserves the original configuration of the biopolymer.

The methods can also include the step of obtaining the broth. For example, the broth may be ordered from a supplier. If the broth is obtained, then preferably the biopolymer is in its original configuration. Any discussion of embodiments above regarding the broth or any ingredient in the broth is meant to apply to a broth that is obtained without the need to repeat all embodiments for the obtained broth. For example, if an embodiment discloses that the microbial cells can be deactivated and/or removed, then the broth obtained can contain deactivated microbial cells or it can be free of microbial cells.

The methods include the step of cryodesiccating the broth. The step of cryodesiccating can include freezing the broth. The broth can be frozen by lowering the temperature of the broth to a temperature below the triple point of the broth. By lowering the temperature of the broth below the triple point ensures that sublimation rather than melting will occur in the drying steps. The triple point of a substance is the temperature and pressure at which the three phases (i.e., gas, liquid, and solid) of that substance coexist in thermodynamic equilibrium. Normally, larger crystals are easier to freeze-dry compared to smaller crystals. In order to produce larger crystals, the broth can be frozen slowly or can be cycled up and down in temperature. The broth can be frozen by a variety of mechanisms, for example by mechanical refrigeration, dry ice and methanol, liquid nitrogen, or using a freeze-drying machine.

The step of cryodesiccating can also include reducing the surrounding pressure of the broth to a pressure less than or equal to the pressure necessary to allow the water of the broth to sublime, wherein the step of reducing is performed after the step of freezing. Sublimation is the process whereby frozen water undergoes a phase change from a solid directly to a gas without proceeding through the intervening liquid phase. The sublimation of the water of the broth can be referred to as drying. There can be a primary and a secondary drying phase. During the primary drying phase, the pressure can be lowered (to the range of a few millibars), and enough heat can be supplied to the material for the water to sublime. The amount of heat necessary can be calculated using the sublimating molecules' latent heat of sublimation. By way of example, the latent heat of sublimation of water is 2,833 Joules per gram of water. In the primary drying phase, approximately 95% of the water in the broth can be sublimated. Primary drying can be a slow process, because, if too much heat is added, the material's structure could be altered. The secondary drying phase can be used to remove unfrozen water molecules from the broth, as ice was removed in the primary drying phase. This part of the freeze-drying process is governed by the material's adsorption isotherms. Adsorption isotherm is the equilibrium of the sorption of a material at the surface boundary of the material at a constant temperature. It represents the amount of material bound at the surface as a function of the material present in the broth. During the secondary drying phase, the temperature is generally higher than the temperature during the primary drying phase. Usually the pressure is also lowered in this phase. According to an embodiment, the final water content in the broth after the step of cryodesiccating is in the range of 0% to about 4%, preferably from 0% to about 2%.

The methods can further include the step of packaging the broth after the step of cryodesiccating the broth. The broth can be vacuum sealed in the package. The methods can further include the step adding the broth to a liquid after the step of cryodesiccating the broth. According to an embodiment, the broth is added to the liquid such that at least one property of the liquid is changed. The property of the liquid that can change includes, without limitation, the viscosity, density, or suspending properties of the liquid. More than one property of the liquid can change. Industries in which the change of a property of a liquid might be desirable include, but are not limited to, oil and gas, food products, pharmaceuticals, water and waste water processing, filtration, waste management, brewing, solids handling, construction, and renewable energy. The biopolymer can be capable of swelling in a liquid. According to an embodiment, the liquid is selected such that the biopolymer is capable of swelling. The liquid can be a water- or hydrocarbon-based liquid.

According to an embodiment, the liquid is a wellbore treatment fluid. The wellbore treatment fluid can be any fluid used to treat at least a portion of a well or subterranean formation. Examples of commonly used wellbore treatment fluids include, but are not limited to, drilling fluids, spacer fluids, frac fluids, acidizing fluids, work-over fluids, completion fluids, stimulations fluids, and cement compositions. The methods can also include the step of introducing the treatment fluid into a well after the step of adding the broth to the liquid. The well can include, without limitation, an oil, gas or water producing well, an injection well, or a geothermal well. As used herein, a “well” includes at least one wellbore. A wellbore can include vertical, inclined, and horizontal portions, and it can be straight, curved, or branched. As used herein, the term “wellbore” includes any cased, and any uncased, open-hole portion of the wellbore. A near-wellbore region is the subterranean material and rock of the subterranean formation surrounding the wellbore. As used herein, a “well” also includes the near-wellbore region. As used herein, “into a well” means and includes into any portion of the well, including into the wellbore or into the near-wellbore region via the wellbore.

Without being limited by theory, it is believed that by cryodesiccating the broth, the original configuration of the biopolymer is preserved. It is also believed that traditional recovery methods including de-watering, precipitating, and drying a biopolymer causes the configuration of the biopolymer to become tangled or randomly coiled. By maintaining the original configuration, the amount of surface area of the biopolymer available to come in contact with a swelling liquid (e.g., a water- or hydrocarbon-based liquid) is greater compared to a biopolymer recovered by traditional methods. By having a greater surface area available to come in contact with the swelling liquid, the biopolymer can swell more quickly and efficiently; and the biopolymer can be added to the swelling liquid at a higher rate compared to a biopolymer recovered by traditional methods. Moreover, the amount and degree of fish eye formation can be substantially reduced or even eliminated.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is, therefore, evident that the particular illustrative embodiments disclosed above may be altered or modified and all such variations are considered within the scope and spirit of the present invention. While compositions and methods are described in terms of “comprising,” “containing,” or “including” various components or steps, the compositions and methods also can “consist essentially of” or “consist of” the various components and steps. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a to b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. If there is any conflict in the usages of a word or term in this specification and one or more patent(s) or other documents that may be incorporated herein by reference, the definitions that are consistent with this specification should be adopted.

What is claimed is:

1. A method of providing a biopolymer comprising:
cryodesiccating a broth, wherein the broth comprises:
(A) a biopolymer, wherein the biopolymer is an exopolysaccharide; and
(B) a liquid growth medium.

2. The method according to claim 1, wherein the biopolymer is a homopolymer or a copolymer.

3. The method according to claim 1, wherein the biopolymer is selected from the group consisting of, acetan, alginate, cellulose, chitosan, curdlan, cyclosophorans, dextran, emulsan, galactoglucopolysaccharides, gellan, glucuronan, N-acetyl-glucosamine, N-acetyl-heparosan, hyaluronic acid, indican, kefiran, lentinan, levan, pullulan, scleroglucan, schizophyllan, stewartan, succinoglycan, xanthan, and welan.

4. The method according to claim 1, wherein the broth contains the biopolymer in its original configuration, wherein the original configuration are long chains.

5. The method according to claim 1, further comprising the step of producing the biopolymer.

6. The method according to claim 5, wherein the biopolymer is produced by the fermentation of at least one carbohydrate via a microbe.

7. The method according to claim 6, wherein the broth is a fermentation broth.

8. The method according to claim 6, wherein the broth further comprises microbial cells and nutrients.

9. The method according to claim 8, further comprising the step of deactivation and/or removal of at least some of the microbial cells in the broth after the production of the biopolymer.

10. The method according to claim 6, wherein the carbohydrate is a monosaccharide or a disaccharide.

11. The method according to claim 10, wherein the monosaccharide is selected from the group consisting of glucose, fructose, galactose, xylose, and combinations thereof.

12. The method according to claim 10, wherein the disaccharide is selected from the group consisting of sucrose, lactulose, lactose, maltose, trehalose, cellobiose, and combinations thereof.

13. The method according to claim 6, wherein the microbe is a bacterium.

14. The method according to claim 13, wherein the bacterium is selected from the group consisting of Acetobacter xylinum, Azotobacter vinelandii, Alcaligenes faecalis var. myxogenes, Agrobacterium spp., Rhizobium spp., Xanthomonas spp., Leuconostoc mesenteroides, Leuconostoc dextranicum, Lactobacillus hilgardii, Acinetobacter calcoaceticus, Achromobacter spp., Agrobacterium radiobacter, Pseudomonas marginalis, Rhizobium spp., Zooglea' spp., Aureomonas elodea, Sphingomonas paucimobilis, Sinorhizobium meliloti, Staphylococcus epidermidis, Escherichia coli, Streptococcus equi, Beijerinckia indica, Alcaligenes viscosus, Zymomonas mobilis, Bacillus subtilis, Pantoea stewartii subsp. stewartii, Xanthomonas campestris, or Alcaligenes spp.

15. The method according to claim 6, wherein the microbe is a fungus.

16. The method according to claim 15, wherein the fungus is selected from the group consisting of Mucorales spp., Lentinus edodes, Aureobasidium pullulans, Sclerotium rolfsii, Sclerotium delfinii, Sclerotium glucanicum, or Schizophylum commune.

17. The method according to claim 1, wherein the step of cryodesiccating comprises freezing the broth.

18. The method according to claim 17, wherein the broth can be frozen by lowering the temperature of the broth to a temperature below the triple point of the broth.

19. The method according to claim 17, wherein the step of cryodesiccating comprises reducing the surrounding pressure of the broth to a pressure less than or equal to the pressure necessary to allow the water of the broth to sublimate, wherein the step of reducing is performed after the step of freezing.

20. The method according to claim 1, further comprising the step of packaging the broth after the step of cryodesiccating the broth.

21. The method according to claim 20, wherein the broth is vacuum sealed in a package.

22. The method according to claim 1, further comprising the step adding the broth to a liquid, wherein the step of adding is performed after the step of cryodesiccating the broth.

23. The method according to claim 22, wherein the liquid is a wellbore treatment fluid.

24. The method according to claim 23, wherein the wellbore treatment fluid is a drilling fluid, a spacer fluid, a frac fluid, an acidizing fluid, a work-over fluid, a completion fluid, a stimulations fluid, or a cement composition.

25. The method according to claim 24, further comprising the step of introducing the treatment fluid into a well, wherein the step of introducing is performed after the step of adding the broth to the liquid.
